(19) United States
(12) Patent Application Publication
(10) Pub. No.: US 2010/0050502 A1
(43) Pub. Date: Mar. 4, 2010

(54) SYSTEMS AND METHODS FOR HYDROTHERMAL CONVERSION OF ALGAE INTO BIOFUEL

(75) Inventors: Benjamin Chiau-pin Wu, San Ramon, CA (US); Charity Ann DeLuca, San Mateo, CA (US); Emma Kathryn Payne, Belmont, CA (US)

Correspondence Address: JONES DAY, 222 EAST 41ST ST, NEW YORK, NY, 10017, US

(73) Assignee: LiveFuels, Inc., San Carlos, CA (US)

(21) Appl. No.: 12/544,862

(22) Filed: Aug. 20, 2009

Related U.S. Application Data
(60) Provisional application No. 61/090,817, filed on Aug. 21, 2008.

(51) Int. Cl. C10L 1/18; C12M 1/00

(52) U.S. Cl. 44/308; 435/289.1

(57) ABSTRACT

The invention provides a system for obtaining biofuel from an algae composition comprising algae and water. The system comprises a pump for pressurizing the algae composition to a predefined pressure and a heater for heating the algae composition to a predefined temperature. Lipids in the algae are extracted and/or hydrolyzed to form fatty acids at a set of predefined temperature and pressure. The water may be in a subcritical or supercritical state at the predefined pressure and predefined temperature. The system further comprises a separator for partitioning the treated algae composition into an organic phase which includes the lipids and/or fatty acids, an aqueous phase, and a solid phase with biomass residue, and for collecting the organic phase. The organic phase can be upgraded to biofuel.

### 1. INTRODUCTION

This application generally relates to systems and methods for producing biofuel from algae.

### 2. BACKGROUND OF THE INVENTION

The United States presently consumes about 42 billion gallons per year of diesel for transportation. In 2007, a nascent biodiesel industry produced 250 million gallons of a bio-derived diesel substitute produced from mostly soybean oil in the U.S. Biodiesel are fatty acid methyl esters (FAME) made typically by the base-catalyzed transesterification of triglycerides, such as vegetable oil and animal fats. Although similar to petrodiesel in many physicochemical properties, biodiesel is chemically different and not fungible with the existing infrastructure. However, a practical and affordable feedstock for biodiesel has yet to be developed. For example, the price of soybean oil has doubled in response to the added demand from the biodiesel industry, thus limiting the growth of the biodiesel industry.

It has been proposed to use algae as a feedstock for producing biofuel, such as, biodiesel. For example, some algae strains can produce up to 50% of their dried body weight in triglyceride oils. Algae also do not need arable land, and can be grown with impaired water, neither of which competes with terrestrial food crops. Moreover, the oil production per acre can be nearly 40 times that of a terrestrial crop, such as soybeans. The present invention provides a cost effective method for converting algae into biofuel.

### 3. SUMMARY

The invention provide systems and methods for hydrothermal conversion of algae into biofuel. In one embodiment of the invention, a system is provided for obtaining biofuel from an algae composition comprising algae and water. In another embodiment of the invention, a method of obtaining biofuel from an algae composition comprising algae and water is provided which comprises treating the algae composition with near-critical or supercritical water. The process can comprise pumping the algae composition up to a predefined pressure and heating the algae composition to a predefined temperature, wherein lipids in the algae are extracted and/or hydrolyzed to form fatty acids. The treatment with near-critical or supercritical water can be repeated, or the treated algae composition can be treated with a temperature, a pressure, and/or an interval that is different from the temperature, pressure, and/or interval of a preceding treatment. The process further comprise separating the algae composition into an organic phase which includes the lipids and/or fatty acids, an aqueous phase, and a solid phase; and collecting the organic phase as biofuel. The organic phase can then be upgraded to either biodiesel or green diesel by transesterification or hydrogenation, respectively. The aqueous and solid phases may be upgraded under a second set of reaction conditions to form biocrude. The invention encompasses the organic phase, the aqueous phase, and the solid phase obtained after the process of the invention, as well as the refined biofuel obtained from the organic phase.

In some embodiments of the invention, the water used in the invention process is in a near-critical state at the predefined pressure and predefined temperature. In some embodiments, the water is in a supercritical state at the predefined pressure and predefined temperature. In some embodiments, the predefined pressure is between 5 atm and 400 atm. In some embodiments, the predefined temperature is between 100° C. and 450° C. or between 325° C. and 425° C. In some embodiments, the lipids include polar lipids and/or neutral lipids. In some embodiments, the second predefined temperature used for conversion to biocrude is above 450° C. In various embodiments, algae in the algae composition belong to the one of the following groups: Scenedesmus, Chlorella, Dunaliella, Spirulina, Coelastrum, Micractinium, Nannochloropsis, Porphyridium, Nostoc, and Haematococcus.

### 4. BRIEF DESCRIPTION OF DRAWINGS

FIG. 1 illustrates a method of obtaining biofuel from algae, according to some embodiments.

FIG. 2 illustrates a plan view of a system for obtaining biofuel from algae, according to some embodiments.

### 5. DETAILED DESCRIPTION OF THE INVENTION

The invention provides systems and methods for hydrothermal conversion of algae into biofuel by the use of near-critical or supercritical water which increase the net amount of useful energy obtainable from algae. To produce biodiesel from algae, the algae are first harvested from an open pond at a concentration of about 0.2 g/L in water. The algae are then sequentially dewatered in several steps typically concluding with centrifugation, which produces an algal paste of about 15% solids. Conventionally, the paste is then fully dried by evaporation. Oil is then extracted from the dried algae with an organic solvent such as hexane, which is then evaporated to leave the residual algal oil, or triglycerides. The term “about,” as used herein, unless otherwise indicated, refers to a value that is no more than 20% above or below the value being modified by the term.

However, such a conventional method for producing biodiesel from algae can be prohibitively expensive. First, the harvested algae is relatively dilute (e.g., about 0.2 g/L) and producing a gallon of oil requires processing 10,000 to 40,000 gallons of water. Because water is heavy, and has a high heat capacity, it can take a large amount of energy to move and to heat such a large volume of water. Indeed, the amount of energy it takes to fully dry an algal paste can be approximately equivalent to the amount of energy that can be obtained from the biofuel product, resulting in essentially no net gain in energy from the algae.

The present invention brings water that is present in an algae composition to a near-critical or supercritical state for use as a solvent to extract lipids. The near-critical or supercritical water can also act as a hydrolyzing agent. The extracted lipids include triglycerides and/or free fatty acids. First, the use of near-critical or supercritical water obviates the energy-intensive step of drying the algae composition completely by evaporation as used in conventional processes. The amount of energy needed to heat and pressurize the water in an algal composition to a near-critical state is significantly lower than the amount of energy that would be needed to vaporize the same amount of water from the composition. For example, boiling water at 100° C. requires 1000 BTU/pound, whereas under a pressure of 80 atm, heating water to 300° C. requires only about 500 BTU/pound, an energy savings of 50%.

Additionally, the solubility and reactivity characteristics of the near-critical or supercritical water allow the water to extract as well as hydrolyze polar and/or non-polar lipids in the algae. Without wishing to be bound to a theory, the hydrolysis of neutral and polar lipids are believed to take place via the following reaction pathways, respectively:

### Hydrolysis of Neutral Lipid into Glycerol and Free Fatty Acids

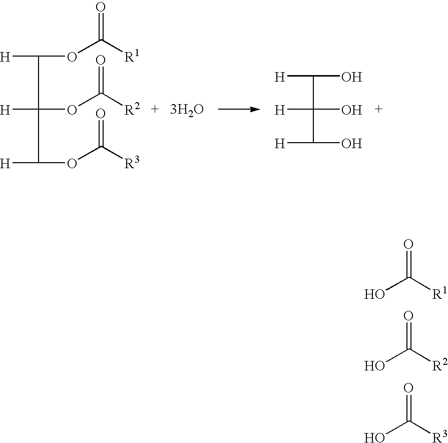

### Hydrolysis of Polar Lipid (e.g., Phospholipid) into Glycerol and Free Fatty Acids

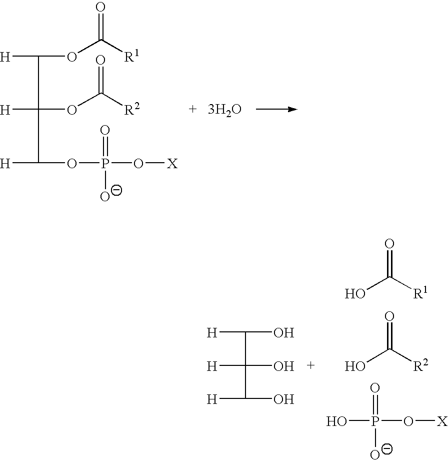

where R¹, R², and R³ are hydrocarbon chains. Some example chains for R¹, R², and R³ can each independently be:

palmitic: —(CH₂)₁₄—CH₃

stearic: —(CH₂)₁₆—CH₃

oleic: —(CH₂)₇CH═CH(CH₂)₇CH₃

linoleic: —(CH₂)₇CH═CH—CH₂—CH═CH(CH₂)₄CH₃

or linolenic: —(CH₂)₇CH═CH—CH₂—CH═CH—CH₂—CH═CH—CH₂—CH₃

and X can be, for example, choline:

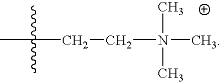

The chains and X can be any naturally occurring moiety in the algal polar or neutral lipids. In certain embodiments of the invention, the polar lipids are part of cell membranes of the algae and contain phosphorous groups, and the near-critical or supercritical water extracts the polar lipids from the cell membranes and hydrolyzes the phosphorous-containing groups.

In contrast, conventional methods of base-catalyzed transesterification of lipids use organic solvents such as methanol, and caustic chemicals, such as NaOH. Typically, these methods have no effect on polar lipids which are chemically inert to transesterification. Because polar lipids can represent a significant portion of the total lipids in the algae, conventional methods that are incapable of converting polar lipids into biofuel produce only a fraction of the energy that can potentially be obtained from the algae. Thus, use of near-critical or supercritical water can potentially increase the useful oil yield from the algae by 100% as compared to conventional lipid extraction.

In some embodiments of the invention, the algae composition is obtained by dewatering algae. The methods of the invention do not require that the algae composition be dried. The algae composition can be obtained from a monoculture, a mixed culture, or a culture where there is one or several predominant species.

The system of invention generally comprises a pump for pressurizing the algae composition to a predefined pressure, a heater for heating the algae composition to a predefined temperature, and a reactor wherein lipids in the algae are extracted and/or hydrolyzed to form fatty acids at the predefined temperature and the predefined pressure. The reactor may comprise an integrated pump and heater to bring the algae composition to the desired temperature and pressure. The system can further comprise a separator for partitioning the algae composition into an organic phase which includes the lipids and/or fatty acids, and an aqueous phase, and for collecting the organic phase. In certain embodiments of the invention, the system further comprises a device for dewatering and a separator/polisher for removing water and other impurities such as phosphorus. In certain embodiments, the system further comprises a centrifuge, a sedimentation tank, a filter, a flocculent, and/or a semi-permeable membrane, or a certain combination of the forgoing, for harvesting the algae. The system can also provide the treatment of the aqueous and/or solid phases at a second predefined temperature and a second predefined pressure; to convert at least a portion of the aqueous and/or solid phases into biocrude.

As used herein the term “biofuel” generally refers to combustible organic liquids derived from biological origin. The term “biocrude” refers to a biofuel that requires further processing or refining before it can be used in conventional combustion processes, e.g. in diesel engines. The term “biodiesel” and “green diesel” refers to refined products that can be used directly by an end-user, such as a motorist. The fuel properties of green diesel are identical to petrodiesel, and therefore it is a completely fungible product.

While the neutral lipids and free fatty acids are useful, high value products that can be used for biocrude, the residue from the hydrothermal processing of the algae can also be further processed to produce additional biofuel feedstocks. For example, in some embodiments, an extract comprising an aqueous phase, an organic phase, and a solid phase is produced by the near- or supercritical water of the process. The organic phase includes neutral lipids and free fatty acids produced by hydrolysis of polar and non-polar lipids, while the aqueous phase and solid phase together include proteins and carbohydrates from the algae, and other substances in the algae composition, collectively referred to herein as process residues. These process residues can readily be converted into additional biocrude by changing the conditions to another temperature and another pressure that is over the critical temperature of water (i.e., 374° C. and 218 atm). The resulting supercritical water can thermochemically (e.g., via hydrolysis and pyrolysis) convert the residue into biocrude.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. Those of skill in the art will be able to practice the systems and methods provided herein using conventional techniques of algae biology, microbiology, chemistry, and chemical engineering, unless otherwise indicated. Conventional techniques are explained fully in the literature. See, e.g., Handbook of Microalgal Culture, edited by Amos Richmond, Blackwell Science, (2004), and Aquaculture. Farming Aquatic Animals and Plants, Editors: John S. Lucas and Paul C. Southgate, Blackwell Publishing, (2003), the entire contents of which are incorporated herein by reference.

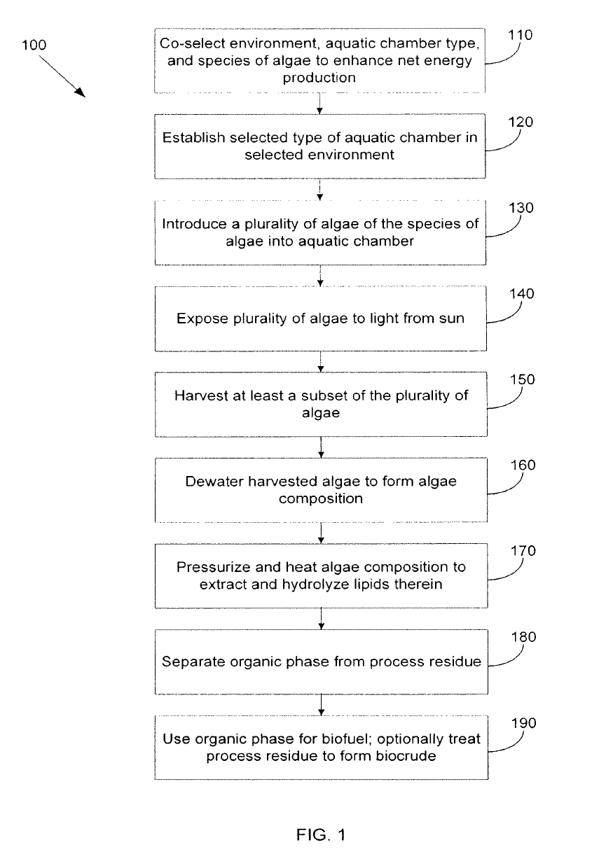

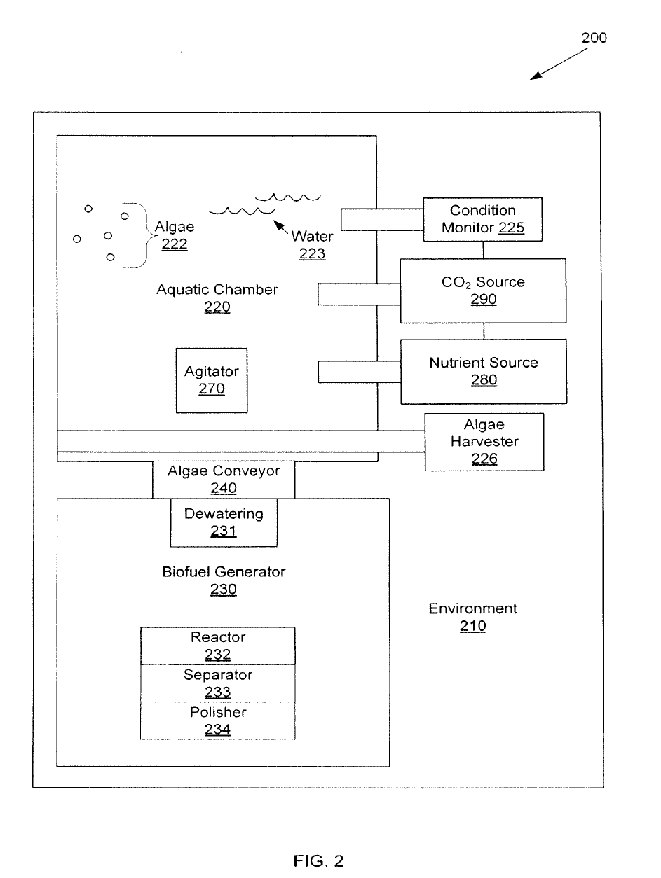

For clarity of disclosure, and not by way of limitation, the detailed description of the invention is divided into the subsections which follow. The hydrothermal conversion processes of the invention are described in details in Section 5.1. FIG. 1 provides an overview of a method 100 of obtaining biofuel from algae, according to certain embodiments of the invention. The algae compositions are described in details in Section 5.2 and an exemplary system of the invention is depicted in FIG. 2. Lipids and biofuels of the invention are described in Section 5.3.

### 5.1. Hydrothermal Conversion

According to the invention, the algae composition is converted into biofuel using a hydrothermal process. The process, which can be a batch process, a continuous process, or a semi-continuous process, comprises pressurizing an algae composition to a predefined pressure above atmospheric pressure, and heating the composition to a predefined temperature, such that water in the composition reaches a near-critical or supercritical state. Close to water's critical point, small changes in pressure or temperature result in large changes in density, allowing the physicochemical properties of water, such as its diffusivity and solvent properties, to be tuned. The near- and supercritical water in the algae composition has significantly different properties than liquid water at ambient conditions. Among other things, the water in the algae composition under process conditions can diffuse through the cell membranes of the algae and dissolve polar and/or neutral lipids within the algae and in the cell membranes of the algae. The water under process conditions can also hydrolyze the polar and/or neutral lipids in the algae and convert it into free fatty acids, which would facilitate either extraction through the cell membranes if the cells are partially intact or significant disruption of the cell membrane. Under certain process conditions of the invention, the algae composition exists in a single phase, in which the aqueous and organic components are miscible with one another.

The term “subcritical” or “near-critical water” refers to water that is pressurized above atmospheric pressure at a temperature between the boiling temperature (100° C. at 1 atm) and critical temperature (374° C.) of water. The term “supercritical water” refers to water above its critical pressure (218 atm) at a temperature above the critical temperature (374° C.). In the methods of the invention, it is preferable to apply a temperature that is below the temperature at which fatty acids in the algae composition are pyrolyzed or gasified into lower molecular weight components. The temperature and pressure used in the invention process maintain the water in the algae in one or more sub-, near- or super-critical state(s), i.e., at an elevated pressure above 1 atm and a temperature between 100° C. and 500° C. The algae composition is held at one or more of the preselected temperature(s) and preselected pressure(s) for an amount of time that facilitates, and preferably maximizes, hydrolysis and/or extraction of various types of lipids. The temperature, pressure, and reaction time are also adjusted during the method such that triglycerides and free fatty acids remain substantially intact. Techniques and equipment for heating and for pressurizing a composition comprising water and solids are well known in the art, and any one or more of such techniques and equipment can be used in the methods of the invention. For example and without limitation, a composition comprising water can be pressurized in a container of constant volume where the composition is heated. In certain embodiments, the methods of the invention can thus comprise heating an algal composition to a predefined temperature in a container with a constant, defined volume, without applying external pressure. In certain embodiments, additional water, such as but not limited to recycled near-critical or supercritical water, is provided to the algae composition.

In various embodiments of the invention, the pressure can be between 5 atm and 400 atm, e.g., between 5 atm and 70 atm, or between 70 atm and 170 atm, or between 170 atm and 400 atm, or about 50, 70, 80, 90, 100, 120, 150, or 200 atm; the temperature is between 100° C. and 500° C., e.g., between 100° C. and 200° C., between 200° C. and 300° C., between 250° C. and 350° C., between 250° C. and 400° C., between 300° C. and 374° C., between 325° C. and 425° C., between 374° C. and 500° C., or about 100° C., 150° C., 200° C., 250° C., 260° C., 270° C., 280° C., 290° C., 300° C. 310° C., 320° C., 330° C., 340° C., 350° C., 360° C., 370° C., 380° C., 400° C. or 500° C. The reaction time or interval is between 5 seconds and 60 minutes, between 1 minute and 20 minutes, between 5 minute and 10 minutes, between 30 seconds and 60 seconds, or about 1, 2, 3, 4, 5, 10, 15, 20, 30, 40, 50 or 60 minutes. For example, in section 6, an algae composition was exposed to a process condition comprising a temperature of about 300° C. at about 80 atm for about 10 minutes. It is contemplated that an algae composition can be treated or exposed to process conditions that are defined by a time series of temperature and pressure set points. In some embodiments of the invention, the process condition is continuously changing, e.g., the time duration can be governed in part by the time required for the reactor to ramp up or down from one temperature and pressure to another desired temperature and/or pressure.

Because various types of lipids produced by algae may hydrolyze and/or pyrolyze at different temperatures, in some embodiments, the process conditions, i.e., the temperature, pressure, and reaction time are selected according to the population or species of algae to enhance the recovery of specific types of lipids, such as, intact neutral lipids and free fatty acids, and to limit degradation of lipids and free fatty acids. The selection of an appropriate set of process conditions, i.e., combinations of temperature, pressure, and process time can be determined, among other things, by assaying the quantity and quality of lipids and free fatty acids produced by a particular species or a population (mixed species) of algae under a variety of process conditions, and using combinations that enhance or maximize, the net yield of desired products from the algae composition, e.g., neutral lipids. Accordingly, the invention provides sets of process conditions wherein the lipids are extracted from the algae before they are hydrolyzed; or in another embodiment, the lipids are hydrolyzed within the algae and the free fatty acids then extracted; or in yet another embodiment, a mixture of non-hydrolyzed lipids and free fatty acids are extracted; or in yet another embodiments, pre-existing free fatty acids are extracted (i.e., hydrolysis is not needed in order to generate these free fatty acids); or in yet another embodiment, polar lipids are converted into free fatty acids; or in yet another embodiment, neutral lipids are extracted intact without hydrolysis; or in yet another embodiment, cell membranes are sufficiently disrupted that the lipids are readily available for extraction or separation. The methods of the invention can comprises subjecting an algae composition to a sequence of process conditions that can bring about one or more of the foregoing reactions. It is contemplated that certain process conditions of the invention can result in substantially complete recovery of free fatty acids, polar lipids, and/or neutral lipids from the algae. Methods and process conditions that result in extraction of about 5%, 10%, 20%, 30%, 40%, 50%, 60%, 70%, 80%, 90%, or 95% of all the lipids in the algae are included. Also encompassed are methods and process conditions that result in extraction of about 5%, 10%, 20%, 30%, 40%, 50%, 60%, 70%, 80%, 90%, or 95% of the total free fatty acids, total polar lipids, or total neutral lipids, in the algae.

Upon cooling, the finally treated algae composition, also referred to as an extract or algae extract, partitions into an aqueous phase, an organic (oily) phase, and a solid phase. The aqueous phase contains residual proteins and carbohydrates from the algae, the organic phase contains the free fatty acids, and the solids phase contains residual solids from the algae, e.g., intact and/or broken cell membranes of the algae. The organic phase can be separated from the aqueous and solid phases and collected using any technique well known in the art, e.g., centrifugation. The organic phase can be used directly as biofuel or be converted to either biodiesel through transesterification, such as base-catalyzed transesterification, or green diesel through hydrocatalytic processing. The organic phase resulting from the hydrothermal process of the invention is a composition encompassed by the invention. Such a composition can be defined by the starting algae composition and the process conditions. The aqueous and/or solid phases resulting from the process, also encompassed by the invention, can be further processed to form biocrude, which can be used, for example, as fuel or as fertilizer. Techniques for transesterification and hydrocatalytic processing are well known in the art and are applicable to convert the organic and aqueous/solid phases into various types and grades of biofuels.

Referring to FIG. 1, first, an environment, an aquatic chamber, and one or more species of algae are selected to enhance energy production from the system (110). For example, the environment and the type of aquatic chamber to be established in that environment are selected to be hospitable to growth of the algae. In some embodiments of the invention, the environment is non-arable land, so as to avoid using land that could otherwise be used for growing food crops.

In various embodiments of the invention, one or more species of algae is selected to be cultured in the aquatic chamber and the environment. The selection is based, in part, on the particular temperature characteristics of the environment, qualities of the water and features of the aquatic chamber. Preferably, the selected alga is the dominant species of algae in the aquatic chamber. The aquatic chamber (130) established in the selected environment (120) is constructed to have a surface area and depth that expose the algae to sunlight for efficient algal growth. The algae can be cultured under light from the sun (140) or artificial light.

After a predefined amount of time (e.g., after the algal population increases to a specified density, or after the population growth rate of the algae drops below a specified value), at least a subset of the plurality of algae are harvested (150). In one example, the algae are harvested using a pump that withdraws the algae-containing water from the aquatic chamber. In some embodiments of the invention, the harvested algae are dewatered (160) using any method known in the art to form an algae composition. The algae composition is then pressurized and heated to extract and hydrolyze the lipids therein (170).

After hydrothermal treatment, the algae extract is allowed to cool, and an organic phase separates from the process residues which include an aqueous phase and optionally also a solid phase (180). The organic phase includes the free fatty acids resulting from the hydrolysis of the algal lipids. The aqueous phase includes residual proteins and carbohydrates from the algae. The solid phase includes residual solid material from the algae, such as cell membranes, which may be intact or may be broken, and may settle to the bottom. The organic phase can be suitably partitioned from the aqueous and solid phases, and suitably collected, such as by one or more techniques known in the art, e.g., by distillation, decanting and/or other suitable fluidic separation and collection.

The organic phase can be used directly as a biofuel (190). In another embodiment, the organic phase is processed resulting in biodiesel, ‘green diesel,’ or other biofuel product. Optionally, the residual aqueous and/or solid phases are further processed into biocrude using techniques known in the art. The conversion of the process residues into biocrude is characterized by a breakdown of large molecules into significantly smaller constituents, e.g., by using high temperatures (for example, above 450° C. or 500° C.). In contrast, the hydrothermal process described herein involves extracting neutral lipids or free fatty acids that are recovered intact.

### 5.2 Algae

As used herein the term “algae” refers to any organisms with chlorophyll and a thallus not differentiated into roots, stems and leaves, and encompasses prokaryotic and eukaryotic organisms that are photoautotrophic or photoauxotrophic. The term “algae” includes macroalgae (commonly known as seaweed) and microalgae. For certain embodiments of the invention, algae that are not macroalgae are preferred. The terms “microalgae” and “phytoplankton,” used interchangeably herein, refer to any microscopic algae, photoautotrophic or photoauxotrophic protozoa, photoautotrophic or photoauxotrophic prokaryotes, and cyanobacteria (commonly referred to as blue-green algae and formerly classified as Cyanophyceae). The use of the term “algal” also relates to microalgae and thus encompasses the meaning of “microalgal.” The term “algal composition” refers to any composition that comprises algae, and is not limited to the body of water or the culture in which the algae are cultivated. An algal composition can be an algal culture, a concentrated algal culture, or a dewatered mass of algae, and can be in a liquid, semi-solid, or solid form. A non-liquid algal composition can be described in terms of moisture level or percentage weight of the solids. An “algal culture” is an algal composition that comprises live algae. The microalgae of the invention are also encompassed by the term “plankton” which includes phytoplankton, zooplankton and bacterioplankton. For certain embodiments of the invention, an algal composition or a body of water comprising algae that is substantially depleted of zooplankton is preferred since many zooplankton consume phytoplankton. However, it is contemplated that many aspects of the invention can be practiced with a planktonic composition, without isolation of the phytoplankton, or removal of the zooplankton or other non-algal planktonic organisms. The methods of the invention can be used with a composition comprising plankton, or an aqueous composition obtained from a body of water comprising plankton.

The algae of the invention can be a naturally occurring species, a genetically selected strain, a genetically manipulated strain, a transgenic strain, or a synthetic algae. Algae from tropical, subtropical, temperate, polar or other climatic regions can be used in the invention. Endemic or indigenous algal species are generally preferred over introduced species where an open culturing system is used. Algae, including microalgae, inhabit all types of aquatic environment, including but not limited to freshwater (less than about 0.5 parts per thousand (ppt) salts), brackish (about 0.5 to about 31 ppt salts), marine (about 31 to about 38 ppt salts), and briny (greater than about 38 ppt salts) environment. Any of such aquatic environments, freshwater species, marine species, and/or species that thrive in varying and/or intermediate salinities or nutrient levels, can be used in the invention. The algae in an algal composition of the invention may contain a mixture of prokaryotic and eukaryotic organisms, wherein some of the species may be unidentified. Fresh water from rivers, lakes; seawater from coastal areas, oceans; water in hot springs or thermal vents; and lake, marine, or estuarine sediments, can be used to source the algae. The algae may also be collected from local or remote bodies of water, including surface as well as subterranean water. The algae in an algal composition of the invention may not all be cultivable under laboratory conditions. It is not required that all the algae in an algal composition of the invention be taxonomically classified or characterized in order to for the composition be used in the present invention. Algal compositions including algal cultures can be distinguished by the relative proportions of taxonomic groups that are present.

One or more species of algae are present in the algal composition of the invention. In one embodiment of the invention, the algal composition is a monoculture, wherein only one species of algae is grown. However, in many open culturing systems, it may be difficult to avoid the presence of other algae species in the water. Accordingly, a monoculture may comprise about 0.1% to 2% cells of algae species other than the intended species, i.e., up to 98% to 99.9% of the algal cells in a monoculture are of one species. In certain embodiments, the algal composition comprise an isolated species of algae, such as an axenic culture. In another embodiment, the algal composition is a mixed culture that comprises more than one species of algae, i.e., the algal culture is not a monoculture. Such a culture can occur naturally with an assemblage of different species of algae or it can be prepared by mixing different algal cultures or axenic cultures. In certain embodiments, the algal composition can also comprise zooplankton, bacterioplankton, and/or other planktonic organisms. In certain embodiments, an algal composition comprising a combination of different batches of algal cultures is used in the invention. The algal composition can be prepared by mixing a plurality of different algal cultures. The different taxonomic groups of algae can be present in defined proportions. The combination and proportion of different algae in an algal composition can be designed or adjusted to yield a desired blend of algal lipids. A microalgal composition of the invention can comprise microalgae of a selected size range, such as but not limited to, below 2000 μm, about 200 to 2000 μm, above 200 μm, below 200 μm, about 20 to 2000 μm, about 20 to 200 μm, above 20 μm, below 20 μm, about 2 to 20 μm, about 2 to 200 μm, about 2 to 2000 μm, below 2 μm, about 0.2 to 20 μm, about 0.2 to 2 μm or below 0.2 μm.

A mixed algal composition of the invention comprises one or several dominant species of macroalgae and/or microalgae. Microalgal species can be identified by microscopy and enumerated by counting, by microfluidics, or by flow cytometry, which are techniques well known in the art. A dominant species is one that ranks high in the number of algal cells, e.g., the top one to five species with the highest number of cells relative to other species. Microalgae occur in unicellular, filamentous, or colonial forms. The number of algal cells can be estimated by counting the number of colonies or filaments. Alternatively, the dominant species can be determined by ranking the number of cells, colonies and/or filaments. This scheme of counting may be preferred in mixed cultures where different forms are present and the number of cells in a colony or filament is difficult to discern. In a mixed algal composition, the one or several dominant algae species may constitute greater than about 10%, about 20%, about 30%, about 40%, about 50%, about 60%, about 70%, about 80%, about 90%, about 95%, about 97%, about 98% of the algae present in the culture. In certain mixed algal composition, several dominant algae species may each independently constitute greater than about 10%, about 20%, about 30%, about 40%, about 50%, about 60%, about 70%, about 80% or about 90% of the algae present in the culture. Many other minor species of algae may also be present in such composition but they may constitute in aggregate less than about 50%, about 40%, about 30%, about 20%, about 10%, or about 5% of the algae present. In various embodiments, one, two, three, four, or five dominant species of algae are present in an algal composition. Accordingly, a mixed algal culture or an algal composition can be described and distinguished from other cultures or compositions by the dominant species of algae present. An algal composition can be further described by the percentages of cells that are of dominant species relative to minor species, or the percentages of each of the dominant species. The identification of dominant species can also be limited to species within a certain size class, e.g., below 2000 μm, about 200 to 2000 μm, above 200 μm, below 200 μm, about 20 to 2000 μm, about 20 to 200 μm, above 20 μm, below 20 μm, about 2 to 20 μm, about 2 to 200 μm, about 2 to 2000 μm, below 2 μm, about 0.2 to 20 μm, about 0.2 to 2 μm or below 0.2 μm. It is to be understood that mixed algal cultures or compositions having the same genus or species of algae may be different by virtue of the relative abundance of the various genus and/or species that are present.

Any one or more methods for dewatering algae can be used, including but not limited to, sedimentation, filtration, centrifugation, flocculation, froth floatation, and/or semipermeable membranes, which can increase the concentration of algae by a factor of about 2, 5, 10, 20, 50, 75, or 100. The dewatering step can be performed serially by one or more different techniques to obtain a concentrated algal composition. See, for example, Chapter 10 in Handbook of Microalgal Culture, edited by Amos Richmond, 2004, Blackwell Science, for description of downstream processing techniques. Centrifugation separates algae from the culture media and can be used to concentrate or dewater the algae. Various types of centrifuges known in the art, including but not limited to, tubular bowl, batch disc, nozzle disc, valve disc, open bowl, imperforate basket, and scroll discharge decanter types, can be used. Filtration by rotary vacuum drum or chamber filter can be used for concentrating fairly large microalgae. Flocculation is the collection of algal cells into an aggregate mass by addition of polymers, and is typically induced by a pH change or the use of cationic polymers. Foam fractionation relies on bubbles in the culture media which carries the algae to the surface where foam is formed due to the ionic properties of water, air and matter dissolved or suspended in the culture media. An algal composition of the invention can be a concentrated algal culture or composition that comprises about 110%, 125%, 150%, 175%, 200% (or 2 times), 250%, 500% (or 5 times), 750%, 1000% (10 times) or 2000% (20 times) the amount of algae in the original culture or in a preceding algal composition. An algal composition can also be described by its moisture level or level of solids, especially when it is in a paste form, such as but not limited to a paste comprising about 1%, 2%, 5%, 10%, 15%, 20%, 25%, 30%, 35%, 40%, 45%, or 50% solids by weight.

It is contemplated that many different algal cultures or bodies of water which comprise plankton, can be used in the methods of the invention. Microalgae are preferably used in many embodiments of the invention; while macroalgae are less preferred in certain embodiments. In specific embodiments, algae of a particular taxonomic group, e.g., a particular genera or species, may be less preferred in a culture. Such algae, including one or more that are listed below, may be specifically excluded as a dominant species in a culture or composition. However, it should also be understood that in certain embodiments, such algae may be present as a contaminant, a non-dominant group or a minor species, especially in an open system. Such algae may be present in negligent numbers, or substantially diluted given the volume of the culture or composition. The presence of such algal genus or species in a culture, composition or a body of water is distinguishable from cultures, composition or bodies of water where such algal genus or species are dominant, or constitute the bulk of the algae. In various embodiments, one or more species of algae belonging to the following phyla can be used in the systems and methods of the invention: Cyanobacteria, Cyanophyta, Prochlorophyta, Rhodophyta, Glaucophyta, Chlorophyta, Dinophyta, Cryptophyta, Chrysophyta, Prymnesiophyta (Haptophyta), Bacillariophyta, Xanthophyta, Eustigmatophyta, Rhaphidophyta, and Phaeophyta. In certain embodiments, algae in multicellular or filamentous forms, such as seaweeds and/or macroalgae, many of which belong to the phyla Phaeophyta or Rhodophyta, are less preferred.

In certain embodiments, the algal composition of the invention comprises cyanobacteria (also known as blue-green algae) from one or more of the following taxonomic groups: Chroococcales, Nostocales, Oscillatoriales, Pseudanabaenales, Synechococcales, and Synechococcophycideae. Non-limiting examples include Gleocapsa, Pseudoanabaena, Oscillatoria, Microcystis, Synechococcus and Arthrospira species.

In certain embodiments, the algal composition of the invention comprises algae from one or more of the following taxonomic classes: Euglenophyceae, Dinophyceae, and Ebriophyceae. Non-limiting examples include Euglena species and the freshwater or marine dinoflagellates.

In certain embodiments, the algal composition of the invention comprises green algae from one or more of the following taxonomic classes: Micromonadophyceae, Charophyceae, Ulvophyceae and Chlorophyceae. Non-limiting examples include species of Borodinella, Chlorella (e.g., C. ellipsoidea), Chlamydomonas, Dunaliella (e.g., D. salina, D. bardawil), Franceia, Haematococcus, Oocystis (e.g., O. parva, O. pustilla), Scenedesmus, Stichococcus, Ankistrodesmus (e.g., A. falcatus), Chlorococcum, Monoraphidium, Nannochloris and Botryococcus (e.g., B. braunii). In certain embodiments, Chlamydomonas reinhardtii are less preferred.

In certain embodiments, the algal composition of the invention comprises golden-brown algae from one or more of the following taxonomic classes: Chrysophyceae and Synurophyceae. Non-limiting examples include Boekelovia species (e.g. B. hooglandii) and Ochromonas species.

In certain embodiments, the algal composition in the invention comprises freshwater, brackish, or marine diatoms from one or more of the following taxonomic classes: Bacillariophyceae, Coscinodiscophyceae, and Fragilariophyceae. Preferably, the diatoms are photoautotrophic or auxotrophic. Non-limiting examples include Achnanthes (e.g., A. orientalis), Amphora (e.g., A. coffeiformis strains, A. delicatissima), Amphiprora (e.g., A. hyaline), Amphipleura, Chaetoceros (e.g., C. muelleri, C. gracilis), Caloneis, Camphylodiscus, Cyclotella (e.g., C. cryptica, C. meneghiniana), Cricosphaera, Cymbella, Diploneis, Entomoneis, Fragilaria, Hantschia, Gyrosigma, Melosira, Navicula (e.g., N. acceptata, N. biskanterae, N. pseudotenelloides, N. saprophila), Nitzschia (e.g., N. dissipata, N. communis, N. inconspicua, N. pusilla strains, N. microcephala, N. intermedia, N. hantzschiana, N. alexandrina, N. quadrangula), Phaeodactylum (e.g., P. tricornutum), Pleurosigma, Pleurochrysis (e.g., P. carterae, P. dentata), Selenastrum, Surirella and Thalassiosira (e.g., T. weissflogii).

In certain embodiments, the algal composition of the invention comprises planktons including microalgae that are characteristically small with a diameter in the range of 1 to 10 μm, or 2 to 4 μm. Many of such algae are members of Eustigmatophyta, such as but not limited to Nannochloropsis species (e.g. N. salina).

In certain embodiments, the algal composition of the invention comprises one or more algae from the following groups: Coelastrum, Chlorosarcina, Micractinium, Porphyridium, Nostoc, Closterium, Elakatothrix, Cyanosarcina, Trachelamonas, Kirchneriella, Carteria, Crytomonas, Chlamydamonas, Planktothrix, Anabaena, Hymenomonas, Isochrysis, Pavlova, Monodus, Monallanthus, Platymonas, Pyramimonas, Stephanodiscus, Chroococcus, Staurastrum, Netrium, and Tetraselmis.

In certain embodiments, any of the above-mentioned genus and species of algae may each be less preferred independently as a dominant species in, or be excluded from, an algal composition of the invention.

FIG. 2 illustrates a plan view of a system 200 for generating biofuel from algae, according to certain embodiments of the invention. System 200 includes environment 210, aquatic chamber 220, and biofuel generator 230. The main source of energy in the system 200 is sunlight, so the environment 210 is selected such that the climate is predominantly sunny. For example, environment 210 is selected to have, on average, greater than 200, greater than 250, greater than 300, or greater than 350 sunny days during the year. Additionally, the environment 210 is selected such that, on average, it does not experience temperatures that are harmful to the development of the algae. For example, the environment can be selected such that, on average, the temperature does not vary by more than about 50° F., or more than about 40° F., or more than about 30° F., or more than about 20° F. over the year. The environment can also, or alternatively, be selected such that, on average, the temperature does not drop below 40° F., below 50° F., below 60° F., or below 70° F. and/or does not rise above 70° F., above 80° F., above 90° F., above 100° F., or above 110° F. over the year. The particular environment and algae species are selected to be compatible with one another. Thus, if a particular constraint (e.g., a financial consideration, or the desire to use non-arable land) requires selection of a particular environment, then the environmental constraint affects the particular species of algae that is selected to be cultured in that environment.

The aquatic chamber 220 is constructed within environment 210. The aquatic chamber 220 contains, among other things, a plurality of algae 222 of the selected species of algae, and water 223. In many embodiments, the aquatic chamber 220 is an “open pond,” meaning that the chamber 220 is exposed directly to the environment 210. In other embodiments (not illustrated), the aquatic chamber 220 is housed in a protective housing that transmits sunlight but at least partially shields the aquatic chamber 220 from the environment 210, prevents other organisms from entering the aquatic chamber, and/or reduces evaporation of water 223.

The aquatic chamber 220 is constructed to expose a relatively large proportion of the algae to sunlight, thus enhancing the growth rate of the algae 222. For example, depending on the concentration of algae 222, light may only penetrate into the top few inches of the water 223 (e.g., the top ¼-4 inches). To prevent algae 222 at the top surface of water 223 from being exposed to too much sunlight, and to expose deeper algae 222 to sunlight, aquatic chamber 220 optionally includes agitator 270 for agitating the algae. Agitator 270 can be any suitable mechanism for agitating the water 223, for example, a mechanical agitator such as a paddle wheel, fluid sprayer, or a fluidic agitator such as a bubbler.

The aquatic chamber 220 can have any suitable construction that is compatible with the sunlight-driven growth and subsequent harvesting of algae 222. For example, the aquatic chamber 220 can be an earthen pond that is dug directly into environment 210 with a lateral area and volume selected to enhance growth of algae 222. Optionally, the aquatic chamber 220 is lined with a material (e.g., polymer sheeting) that discourages leakage of water 223 from the chamber and/or discourages the growth of organisms that are detrimental to the growth of algae 222. Alternately, the chamber can be formed of cement or other suitable, water-tight material.

The aquatic chamber 220 is constructed to retain water 223 having characteristics selected to support growth of algae 222. For example, water 223 can be fresh water, brackish water, salt water, or brine, depending on the particular species of algae 222 to be grown therein. As used herein, fresh water is considered to have less than 0.5 parts per thousand (ppt) of dissolved salts; brackish water to have between 0.5 and 35 ppt of dissolved salts; salt water to have between 35 and 50 ppt of dissolved salts; and brine to have greater than 50 ppt of dissolved salts. The pH of water 223 can be selected in order to enhance growth of the algae 222, e.g., from pH 5 to pH 10.

System 200 includes a water condition monitor 225 that monitors the condition of water 223, e.g., monitors the temperature, pH, alkalinity, and concentration of substances such as CO₂, O₂, nitrates, ammonia, phosphorous, other dissolved salt, and/or algae 222 in the water 223. Optionally, water condition monitor 225 is in operable communication with CO₂ source 290 and nutrient source 280, and controllably releases CO₂ and/or nutrients into water 223 as needed in order to maintain the appropriate level of substances in the water 223. Water condition monitor 225 includes one or more suitable sensors and logic for reading the output of the sensor(s), determining whether the sensors indicate suitable substance levels, and controlling CO₂ source 290 and nutrient source 280 as needed to adjust the levels of substances in the water 223.

For example, water condition monitor 225 is operable to control CO₂ source 290 to introduce additional CO₂ into the water 223. As algae 222 photosynthesize, they consume CO₂ in the water and produce O₂. Dissolved levels of CO₂, as either molecular CO₂ or carbonates, may not be sufficient to sustain the optimal growth rate of algae 222. If the CO₂ were to drop below an acceptable level of CO₂ for algal growth, then algal growth would be restricted, thus reducing the formation of algal lipids and also potentially de-equilibrating the ecosystem in aquatic chamber 220. Sources of CO₂ include, but are not limited to, waste CO₂ from industrial processes (such as power generation), or geothermal wells. A source of waste CO₂ is particularly useful for supplementing CO₂ levels in water 223 because it has essentially no financial or energy cost, since it would have otherwise gone to waste, and it also prevents that CO₂ from instead being emitted into the air. Moreover, capturing the CO₂ may soon be monetized through “cap-and-trade” schemes that are already practiced in the Europe and proposed in the U.S., providing for another revenue stream. The CO₂ can be bubbled into water 223, or otherwise suitably introduced.

Water condition monitor 225 is also operable to control nutrient addition 280 into the water 223. Although the algae 222 grow primarily based on energy from the sun, they will need additional elements such as nitrogen and phosphorous in order to grow and reproduce. Nutrient source 280 includes any supplemental nutrients the algae 222 need in order to grow and reproduce. Generally, adding fresh high-protein meal directly to chamber 220 would reduce the net energy produced by the system 200 because that meal would have to be specifically produced for such a purpose, which would require energy and thus reduce the net energy gain from system 200. Nitrogen and phosphorous are useful nutrients to be included in nutrient source 280. Other examples of suitable nutrient sources include dairy farm waste, hog farm waste, human waste, farm runoff, and combinations thereof.

The amount of biofuel that can be produced from the algae 222 is, in part, based on the amount of lipids in the algae (e.g., fats and oils). The algae 222 can have a lipid content of, for example, greater than 5%, greater than 10%, greater than 15%, greater than 20%, greater than 25%, greater than 30%, greater than 35%, or more. The algae 222 can be present in a concentration of between about 200-1000 mg/L of water 223.

The selected species of algae 222 is autotrophic, that is, the algae obtain their energy from the sun. Thus, substantially no additional energy need be input to the system in order to grow the algae 222 (noting that substances and/or nutrients useful for algal growth can, in some embodiments, be added without requiring significant energy to be expended). The species of algae 222 is selected to have a growth rate and a reproduction rate that efficiently produces energy during a predetermined time period, e.g., a 1-10 day period in which the algae are grown in the aquatic chamber.

The algae 222 may be a monoculture (all the same species), or may be a mixture of different species of algae. In an open pond, mixtures of different species of algae tend to grow, often with one dominant species. Therefore, it can be useful to select the algae 222 to be the dominant algal species in the particular environment, even if other algae species are purposefully or incidentally introduced. Some examples of suitable algae that can be used include, but are not limited to: Scenedesmus, Chlorella, Dunaliella, Spirulena, Coelastrum, Micractinium, Euglena, and Cyanobacteria.

System 200 includes an algae harvester 226 for harvesting the algae 222, an algae conveyor 240 for transporting the harvested algae, and a biofuel generator 230 for generating biofuel from the algae. The algae harvester 226 can be any suitable device that allows the algae 222 to be obtained from aquatic chamber 220 at a desired time. For example, in some embodiments, algae harvester 226 is configured to harvest algae 222 mechanically, fluidically, electrically, or using any other suitable harvesting mechanism. In one embodiment, algae harvester 226 is a pump that withdraws water 223 and algae 222 from aquatic chamber 220.

Algae harvester 226 collects algae 222 and water 223 into algae conveyor 240, which transports the algae and water to biofuel generator 230, which may or may not be located adjacent to aquatic chamber 220. In embodiments where biofuel generator 230 is co-located adjacent aquatic chamber 220, the algae conveyor 240 can be, for example, a pipe that feeds algae 222 and water 223 into biofuel generator 230. In embodiments where biofuel generator 230 is located remotely from aquatic chamber 220, the algae conveyor can be, for example, a truck, train, or barge configured to contain the algae 222 and water 223 and to transport them to the biofuel generator 230.

In the illustrated embodiment, the biofuel generator 230 includes a device 231 for dewatering the harvested algae 222, and a reactor 232 for generating biofuel from the algae 222. The reactor comprises a source of reactor pressure, e.g., a liquid feed pump, and a source of heat, e.g. a heater that burns biofuel. Any source of pressure and heat can be used. In other embodiments (not shown) the device 231 is located separately from the reactor 232 and the system includes a conveyor for transporting concentrated algae from the concentrator 231 to the reactor 232. The device 231 increases the concentration of algae 222 in water 223, for example, by a factor of 10 or more (e.g., by a factor of 10 to 100). The device 231 includes any suitable subsystem for increasing the concentration of the algae, e.g., a sedimentation tank, a filter, a flocculent, or a semipermeable membrane for dewatering the harvested algae. The device can also, or alternatively, include a centrifuge for dewatering the harvested algae.

Following concentration, the algae composition is then introduced into reactor 232. Here, the algae composition is subjected to an elevated pressure and a temperature between 100° C. and 500° C. The pressure and temperature together are sufficient to hydrolyze some or all of the lipids in the algae into free fatty acids and to extract the lipids and/or free fatty acids from the algae but preferably without breaking the free fatty acid chains. The reactor 232 can be a closed vessel into which different batches of algae composition are introduced and processed, or can be an open reactor that is configured to continuously process algae composition flowing therethrough.

After the reactor 232 processes the algae composition, the treated algae composition and reaction products partition as the mixture cools into three phases, an aqueous phase, an organic phase, and a solid phase. The organic phase includes free fatty acids resulting from the hydrolysis of the polar and/or neutral lipids in the algae and in certain embodiments, lipids extracted from the algae, while the aqueous and solid phases contain process residues. The reactor 232 may include a separator 233 for partitioning the aqueous and solid phases from the organic phase. The separator can be any suitable mechanical, fluidic, or other type of subsystem for separating the aqueous phase from the organic phase. The separator may be a standalone device fluidically connected to the reactor. For example, the separator can include a fluidic pathway for decanting the phase of lower density (e.g., the organic phase) from above the phase of higher density (e.g., the aqueous and solid phase). Or, for example, the separator can include a fluidic pathway for withdrawing the phase of higher density from below the phase of lower density. In other embodiments, the organic, aqueous, and solid phases are separated using distillation. In some embodiments, the separator is configured to leave the aqueous and solid phases within reactor 232 for further processing into biocrude, while removing the organic phase from reactor 232 for use as biofuel, optionally following further processing. In other embodiments, the aqueous and/or solid phases are subsequently processed into methane using a conventional anaerobic process. In yet another embodiment, the aqueous and/or solid phases can be used as fertilizers.

### 5.3 Lipids and Biofuel

The invention provides a biofuel, a biodiesel, or an energy feedstock comprising lipids derived from algae. Lipids extracted from algae can be subdivided according to polarity: neutral lipids and polar lipids. The major neutral lipids are triglycerides, and free saturated and unsaturated fatty acids. The major polar lipids are acyl lipids, such as glycolipids and phospholipids. A composition comprising lipids and/or hydrocarbons can be described and distinguished by the types and relative amounts of key fatty acids and/or hydrocarbons present in the composition.

Fatty acids are identified herein by a first number that indicates the number of carbon atoms, and a second number that is the number of double bonds, with the option of indicating the position of the double bonds in parenthesis. The carboxylic group is carbon atom 1 and the position of the double bond is specified by the lower numbered carbon atom. For example, linoleic acid can be identified by 18:2 (9, 12).

Algae produce mostly even-numbered straight chain saturated fatty acids (e.g., 12:0, 14:0, 16:0, 18:0, 20:0 and 22:0) with smaller amounts of odd-numbered acids (e.g., 13:0, 15:0, 17:0, 19:0, and 21:0), and some branched chain (iso- and anteiso-) fatty acids. A great variety of unsaturated or polyunsaturated fatty acids are produced by algae, mostly with C₁₂ to C₂₂ carbon chains and 1 to 6 double bonds, mainly in cis configurations. Fatty acids produced by the cultured algae of the invention comprise one or more of the following: 12:0, 14:0, 14:1, 15:0, 16:0, 16:1, 16:2, 16:3, 16:4, 17:0, 18:0, 18:1, 18:2, 18:3, 18:4, 19:0, 20:0, 20:1, 20:2, 20:3, 20:4, 20:5, 22:0, 22:5, 22:6, and 28:1 and in particular, 18:1(9), 18:2 (9, 12), 18:3(6, 9, 12), 18:3(9, 12, 15), 18:4(6, 9, 12, 15), 18:5(3, 6, 9, 12, 15), 20:3(8, 11, 14), 20:4(5, 8, 11, 14), 20:5(5, 8, 11, 14, 17), 20:5(4, 7, 10, 13, 16), 20:5(7, 10, 13, 16, 19), 22:5(7, 10, 13, 16, 19), 22:6(4, 7, 10, 13, 16, 19).

The hydrocarbons present in algae are mostly straight chain alkanes and alkenes, and may include paraffins and the like having up to 36 carbon atoms. The hydrocarbons are identified by the same system of naming carbon atoms and double bonds as described above for fatty acids. Non-limiting examples of the hydrocarbons are 8:0, 9,0, 10:0, 11:0, 12:0, 13:0, 14:0, 15:0, 15:1, 15:2, 17:0, 18:0, 19:0, 20:0, 21:0, 21:6, 23:0, 24:0, 27:0, 27:2(1, 18), 29:0, 29:2(1, 20), 31:2(1, 22), 34:1, and 36:0.

Examples of systems and methods for processing lipids such as algal oil into biofuel, can be found in the following patent publications, the entire contents of each of which are incorporated by reference herein: U.S. Patent Publication No. 2007/0010682, entitled “Process for the Manufacture of Diesel Range Hydrocarbons;” U.S. Patent Publication No. 2007/0131579, entitled “Process for Producing a Saturated Hydrocarbon Component;” U.S. Patent Publication No. 2007/0135316, entitled “Process for Producing a Saturated Hydrocarbon Component;” U.S. Patent Publication No. 2007/0135663, entitled “Base Oil;” U.S. Patent Publication No. 2007/0135666, entitled “Process for Producing a Branched Hydrocarbon Component;” U.S. Patent Publication No. 2007/0135669, entitled “Process for Producing a Hydrocarbon Component;” and U.S. Patent Publication No. 2007/0299291, entitled “Process for the Manufacture of Base Oil.”

### 6. EXAMPLE

The present invention may be better understood by reference to the following non-limiting example, which is provided only as exemplary of the invention. The example should in no way be construed as limiting the broader scope of the invention.

In this experiment, the performance of an exemplary hydrothermal process in extracting lipids from an algal composition was evaluated against a conventional process. Nannochloropsis was chosen as a good representative because of its potentially high productivity and high lipid content, coupled with a robust cell membrane and relatively small size (about 2 to 5 μm). The starting material was a 15% solid/85% moisture algae paste that was produced by centrifugation of an algal culture. Both transesterification in acid-catalyst and organic solvent-based extractions were carried out to determine the total recoverable lipid yield and the distribution amongst three major classes of lipids; neutral lipids (NL), free fatty acids (FFA), and phospholipids (PL). Either n-Hexane or hexane:isopropanol 3:2 (v/v) (HIP) was used to extract each sample. Aminopropyl bonded silica solid phase extraction (SPE) columns were used to separate lipid extracts (both crude and washed) into fractions corresponding to NL, FFA, and PL. All treatments of the algae paste (15% solid) were tested in duplicate and analyzed in triplicate.

According to an embodiment of the invention, one batch of the algae paste was treated at 300° C. for 10 minutes under nominally 80 atm pressure in microreactors comprised of high-pressure tubing and fittings (referred to herein as “treated algae”). A second batch of the algae paste was dried overnight in a vacuum oven at 100° C. (referred to herein as “dried algae”). An untreated third batch of the algae paste was used as a control (referred to herein as “wet algae”).

Wet algae and treated algae were extracted with HIP. Dry algae was extracted with n-hexane or HIP for 18 hrs in a stirred reactor at 60° C. The extraction method is adapted from Hara & Radin (1978, Anal Biochem. 90(1):420-6) and butylated hydroxytoluene (BHT) was used as an antioxidant during the extraction. A sample of dried algae was transesterified with acid catalyst to verify data obtained by gravimetric analysis which essentially converts all lipids into fatty acid methyl esters, and provided an estimate of the maximum theoretical yield.

Certain algae samples were homogenized in about 10 ml of HIP or n-hexane for 3 minutes. The homogenate was centrifuged at 500 g for 5 minutes to separate solids which was re-extracted once with 2 ml of additional solvent. The separated solvent was washed by vortexing with 6 ml of a sodium sulfate (Na₂SO₄) solution (1 g in 15 ml) to remove non-lipids. The mixture was centrifuged at 500 g for 3 minutes. The upper layer that contains extracted lipids was collected, dried for 8 hours in a vacuum manifold unit with nitrogen at a low flow rate. The lipids were dissolved in 150 μl of hexane:chloroform:methanol (95:3:2) with BHT for analysis or stored frozen.

For SPE analysis, the extracted lipids (150 μl) were loaded into a SPE aminopropyl column that had been washed with 8 ml of hexane. The column was eluted first with two loads of chloroform (2.5 ml each). The eluate was collected and labeled Fraction I. The column was then eluted with two loads of ethyl ether with 2% acetic acid (2.5 ml each), and the eluate was collected and labeled Fraction II. The column was finally eluted with two loads of methanol:chloroform (6:1) with 0.05 M sodium acetate and the eluate was collected and labeled Fraction III. All fractions were dried under nitrogen.

Table 1 shows the yields of crude lipid extracts (CLE) from samples of wet algae, dried algae and treated algae by gravimetric and Gas Chromatographic with Flame Ionization Detection (GC-FID) analyses. The GC-FID analysis provides quantitative amounts of lipids that could be identified and characterized. The difference between the two techniques is attributable to lipids that were either unidentified or not eluted during gas chromatography.

| | | Yield of CLE | Yield of CLE |
|---|---|---|---|
| Algae Samples | Solvent | (gravimetric) | (GC-FID) |
| Dried | HIP | 24% | 7% |
| Dried | n-hexane | 18% | 6% |
| Dried | Direct to FAMEs | 18% | 9% |
| Wet | HIP | 13% | 4% |
| Wet | n-hexane | 4% | 1% |
| Treated | HIP | 18% | 2% |
| Treated & | HIP | 18% | 3% |
| homogenized | | | |

The gravimetric data show that hydrothermal processing at 300° C. and 10 min was almost as effective at extracting lipids from Nannochloropsis as the conventional process. The yield by hydrothermal processing followed by extraction with HIP was 18% of total lipids recovered from algae on a dry weight basis, as compared to a yield of 18% (n-hexane) and 24% (HIP) from the conventional process. HIP is known to extract non-lipids from the algae, especially pigments, so typically yields are higher since non-lipids are included. Notably, the extraction of lipids by hydrothermal processing is apparently near-complete. A critical difference is that the conventional process requires both drying of the algae and cell disruption (homogenized), both of which steps are cost-prohibitive. For treated algae, the benefit of adding the homogenizing step is negligible indicating that the cell membranes were already substantially disrupted. Extraction from wet algae was consistently less effective than using dried algae or treated algae.

Table 2 shows the distribution of lipids as percentages of the total recovered lipids from SPE analysis (Fraction I=neutral lipids; Fraction II=free fatty acids; Fraction III=polar lipids).

| | Fraction | | |
|---|---|---|---|
| Treatment | I | II | III |
| Dried algae Extracted with HIP | 31% | 27% | 42% |
| Dried algae Extracted with Hexane | 39% | 31% | 30% |
| Wet algae Extracted with HIP | 10% | 47% | 44% |
| Treated algae Extracted with HIP | 33% | 65% | 2% |
| Treated algae Extracted with HIP (duplicate) | 40% | 57% | 2% |

The data in Table 2 show that the hydrothermal process apparently converted polar lipids to free fatty acid, i.e., polar lipids (Fraction III) decreasing from about 30% to about 2% of total lipids, with a commensurate increase in free fatty acids (Fraction II) from about 30% to about 60%. Since polar lipids are not acceptable feedstock for renewable diesel production, hydrothermal processing would increase the fuel feedstock yield by 30% from Nannochloropsis culture.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed:

1. A method for producing biofuel from an algae composition comprising algae and water, said method comprising treating the algae composition with near-critical water and/or supercritical water, and separating from treated algae composition an organic phase which comprises lipids that are used as biofuel.

2. The method of claim 1, wherein said treating step comprises pressurizing the algae composition to a pressure that is above atmospheric pressure and heating the algae composition to a temperature that is above 100° C., for an interval, such that lipids are extracted from algae in the algae composition.

3. The method of claim 2, wherein said treating step is repeated at least once with a temperature, a pressure, and/or an interval that is different from the temperature, pressure, and/or interval of a preceding treating step.

4. The method of claim 1, wherein said separating step comprises partitioning said treated algae composition into the organic phase and (i) an aqueous phase comprising solids or (ii) an aqueous phase and solid phase.

5. The method of claim 2, wherein the pressure is between 5 atm and 400 atm.

6. The method of claim 2, wherein the temperature is between 100° C. and 450° C.

7. The method of claim 2, wherein the temperature is between 250° C. and 374° C.

8. The method of claim 2, wherein the pressure is 80 atm and the temperature is 300° C.

9. The method of claim 2, wherein the interval is between 30 seconds to 30 minutes.

10. The method of claim 2, wherein polar lipids in said lipids are hydrolyzed to form fatty acids.

11. The method of claim 2, wherein said algae composition comprises 15% solids by weight.

12. The method of claim 2, wherein said method further comprises dewatering said algae composition prior to said treating step.

13. The method of claim 2, further comprising subjecting the organic phase to transesterification or hydrogenation.

14. The method of claim 2, further comprising removing water and/or phosphorous from the organic phase.

15. The method of claim 4, further comprising treating the aqueous phase and/or solid phase at a second temperature and a second pressure, wherein at least a portion of the aqueous phase and/or solid phase is converted into biocrude.

16. The method of claim 15, wherein the second temperature is above 450° C.

17. The method of claim 2, wherein said algae composition comprises algae that belong to at least one group consisting of: Scenedesmus, Chlorella, Dunaliella, Spirulina, Coelastrum, Micractinium, Nannochloropsis, Porphyridium, Nostoc, and Haematococcus.

18. A system for producing biofuel from an algae composition comprising algae and water, said system comprising a reactor for treating said algae composition with near-critical and/or supercritical water, wherein lipids are extracted from algae in said algae composition; and a separator for separating an organic phase from the treated algae composition, wherein said organic phase comprises lipids that are used as biofuel.

19. The system of claim 18, wherein said reactor comprises a heater for heating the algae composition.

20. The system of claim 18, wherein said reactor comprises a pump for pressurizing the algae composition.

21. The system of claim 18, further comprising an algae conveyor and/or a dewatering device.

22. The system of claim 18, further comprising a polisher for removing water and impurities from the organic phase.

23. A composition comprising lipids, said lipids being present in the organic phase that is produced by the method of claim 1.

24. A composition comprising lipids, said lipids being present in the organic phase that is produced by the method of claim 2.

25. A composition comprising lipids, said lipids being present in the organic phase that is produced by the method of claim 8.
